(19) United States
(12) Patent Application Publication
(10) Pub. No.: US 2025/0372236 A1
(43) Pub. Date: Dec. 4, 2025

(54) DEVICES AND METHODS FOR TRAINING SAMPLE CHARACTERIZATION ALGORITHMS IN DIAGNOSTIC LABORATORY SYSTEMS

(71) Applicant: Siemens Healthcare Diagnostics Inc., Tarrytown, NY (US)

(72) Inventors: Yao-Jen Chang, Princeton, NJ (US); Nikhil Shenoy, West Windsor, NJ (US); Ramkrishna Jangale, Baner, Pune (IN); Benjamin S. Pollack, Jersey City, NJ (US); Ankur Kapoor, Plainsboro, NJ (US)

(73) Assignee: Siemens Healthcare Diagnostics Inc., Tarrytown, NY (US)

(21) Appl. No.: 18/876,718

(22) PCT Filed: Jul. 13, 2023
(86) PCT No.: PCT/US2023/027679

§ 371 Date: Dec. 19, 2024

Related U.S. Application Data
(60) Provisional application No. 63/368,456, filed on Jul. 14, 2022.

(51) Int. Cl. G16H 30/40; B25J 9/16; G06V 10/141; G06V 10/25; G06V 20/70; G16H 30/20

(52) U.S. Cl. CPC G16H 30/40; B25J 9/1612; G06V 10/141; G06V 10/25; G06V 20/70; G16H 30/20

(57) ABSTRACT

A method of updating training of an annotation generator of a diagnostic laboratory system includes providing an imaging device in the diagnostic laboratory system, wherein the imaging device is controllably movable within the diagnostic laboratory system; capturing a first image within the diagnostic laboratory system using the imaging device, the first image captured with at least one imaging condition; performing an annotation of the first image using the annotation generator to generate a first annotated image; and updating training of the annotation generator using the first annotated image. Other methods and systems are disclosed.

### CROSS REFERENCE TO RELATED APPLICATION

This application claims the benefit of U.S. Provisional Patent Application No. 63/368,456, entitled “DEVICES AND METHODS FOR TRAINING SAMPLE CHARACTERIZATION ALGORITHMS IN DIAGNOSTIC LABORATORY SYSTEMS” filed Jul. 14, 2022, the disclosure of which is hereby incorporated by reference in its entirety for all purposes.

### FIELD

Embodiments of the present disclosure relate to devices and methods for training sample characterization algorithms in diagnostic laboratory systems.

### BACKGROUND

Diagnostic laboratory systems conduct clinical chemistry or assays to identify analytes or other constituents in biological samples such as blood serum, blood plasma, urine, interstitial liquid, cerebrospinal liquids, and the like. The samples may be received in and/or transported throughout laboratory systems in sample containers. Many of the laboratory systems process large volumes of sample containers and the samples contained in the sample containers.

Some laboratory systems use machine vision and machine learning to facilitate sample processing and sample container identification, which may be based on characterization and/or classification of the sample containers. For example, vision-based machine learning models (e.g., artificial intelligence (AI) models) have been adapted to provide fast and noninvasive methods for sample container identification and characterization. However, the training cost for supporting new types of sample containers or new imaging conditions with the machine learning models can be excessive because large amounts of training data are required to retrain or adapt the machine learning models to characterize new types of sample containers or adapt the machine leaning models to work under new imaging conditions. Therefore, a need exists for laboratory systems and methods that improve training of machine vision systems in laboratory systems.

### SUMMARY

According to a first aspect, a method of updating training of a sample characterization algorithm of a diagnostic laboratory system is provided. The method includes providing an imaging device in the diagnostic laboratory system, wherein the imaging device is controllably movable within the diagnostic laboratory system; capturing a first image within the diagnostic laboratory system using the imaging device, the first image captured with an imaging condition; performing an annotation of the first image using an annotation generator of the diagnostic laboratory system to generate a first annotated image; and updating training of the annotation generator using the first annotated image.

In another aspect, a method of training a sample characterization algorithm of a diagnostic laboratory system is provided. The method includes providing an imaging device in the diagnostic laboratory system, wherein the imaging device is controllably movable within the diagnostic laboratory system; capturing a first image of a sample container using the imaging device, the first image captured with a first imaging condition; performing an annotation of the first image to generate a first annotated image; altering the first imaging condition to a second imaging condition; capturing a second image of the sample container using the imaging device with the second imaging condition; performing the annotation of the second image to generate a second annotated image; training an annotation generator of the diagnostic laboratory system using at least the first annotated image and the second annotated image; altering the second imaging condition to a third imaging condition; capturing a third image of the sample container using the imaging device with the third imaging condition; performing the annotation of the third image using the annotation generator to generate a third annotated image; and further training the annotation generator using at least the third annotated image.

In a further aspect, a diagnostic laboratory system is provided that includes (1) an imaging device controllably movable within the laboratory system, wherein the imaging device is configured to capture images within the laboratory system under different imaging conditions; (2) a processor coupled to the imaging device; (3) a memory coupled to the processor, wherein the memory includes an annotation generator trained to annotate images captured by the imaging device, the processor further including computer program code that, when executed by the processor, causes the processor to (a) receive first image data of a first image captured by the imaging device using at least one imaging condition; (b) cause the annotation generator to perform an annotation of the first image to generate a first annotated image; and (c) update training of the annotation generator using the first annotated image.

Still other aspects, features, and advantages of this disclosure may be readily apparent from the following description and illustration of a number of example embodiments, including the best mode contemplated for carrying out the disclosure. This disclosure may also be capable of other and different embodiments, and its several details may be modified in various respects, all without departing from the scope of the disclosure.

### BRIEF DESCRIPTION OF THE DRAWINGS

The drawings, described below, are provided for illustrative purposes, and are not necessarily drawn to scale. Accordingly, the drawings and descriptions are to be regarded as illustrative in nature, and not as restrictive. The drawings are not intended to limit the scope of the disclosure in any way.

FIG. 1 illustrates a block diagram of a diagnostic laboratory system including a sample handler according to one or more embodiments.

FIG. 2 illustrates a top plan view of an interior of a sampler handler of a diagnostic laboratory system according to one or more embodiments.

FIGS. 3A-3C illustrate different types of sample containers including caps affixed to tubes that may be used within a diagnostic laboratory system according to one or more embodiments.

FIGS. 4A-4C illustrate different types of tubes of sample containers that may be used within a diagnostic laboratory system according to one or more embodiments.

FIG. 5 illustrates a perspective view of a robot in a sample handler of a diagnostic laboratory system coupled to a gantry that is configured to move the robot and an attached imaging device along x, y, and z axes according to one or more embodiments.

FIG. 6 illustrates a side elevation view of the robot of FIG. 5 wherein the imaging device is operative to capture images of a sample container according to one or more embodiments.

FIG. 7 illustrates a side elevation view of the robot of FIG. 6 with a gripper pivotally coupled to a main structure of the robot according to one or more embodiments.

FIG. 8 illustrates a flowchart of a sample container characterization workflow that may be implemented in a characterization algorithm of a diagnostic laboratory system according to one or more embodiments.

FIG. 9 illustrates a workflow used to generate image data with variations used to train characterization algorithms of diagnostic laboratory systems according to one or more embodiments.

FIG. 10 illustrates a flowchart of another example method of updating training of an annotation generator of a diagnostic laboratory system according to one or more embodiments.

FIG. 11 illustrates a flowchart of training an annotation generator of a diagnostic laboratory system according to one or more embodiments.

FIGS. 12A-12I illustrate example images and image annotations in accordance with one or more embodiments.

FIGS. 13A-13F illustrate additional example images and image annotations in accordance with one or more embodiments.

### DETAILED DESCRIPTION

As described, diagnostic laboratory systems conduct clinical chemistry and/or assays to identify analytes or other constituents in biological samples such as blood serum, blood plasma, urine, interstitial liquid, cerebrospinal liquids, and the like. The samples are collected in sample containers and then delivered to a diagnostic laboratory system. The sample containers are subsequently loaded into a sample handler of the laboratory system. The sample containers are then transferred to sample carriers by a robot, wherein the sample carriers transport the sample containers to instruments and components of the laboratory system where the samples are processed and analyzed.

Diagnostic laboratory systems may use vision systems to capture images of sample containers and/or the contents (e.g., biological samples) of the sample containers. The captured images are then used to identify the sample containers and/or the contents of the sample containers. For example, diagnostic laboratory systems may include vision-based AI models configured to provide fast and noninvasive methods for sample container characterization or classification. The AI models may be trained to annotate images of different types of sample containers and variations of tube portions and/or caps of the sample containers. The annotated images may then be used for sample identification purposes.

As new types of sample containers are introduced into diagnostic laboratory systems, the employed AI models must be updated or “trained” to be able to annotate the new sample container types. Retraining the AI models in conventional diagnostic laboratory systems is costly and time consuming because a plurality of different types of sample containers need to be imaged and manually annotated to retrain the AI models. AI models used in machine vision systems are typically trained using images of samples and/or sample containers captured under ideal circumstances, such as in studio-like settings with ideal imaging conditions. Capturing the images under these ideal circumstances is expensive and time consuming. In addition, these ideal conditions rarely exist within the confines of “deployed” laboratory systems. Thus, machine vision systems may not be accurately trained due to the discrepancy between images used to train the machine vision systems and the actual images captured during use of the machine vision systems within deployed diagnostic laboratory systems. Therefore, a need exists for systems and methods that improve training of machine vision systems in diagnostic laboratory systems.

Embodiments of the systems and methods described herein overcome the problems with training sample container identification and classification AI models by capturing training images of the sample containers under actual conditions within a deployed laboratory systems and, in some cases, automatically annotating those training images. The training images may be used to train or retrain AI models (e.g., annotation generators) and the like in the diagnostic laboratory system.

In some embodiments, diagnostic laboratory systems and methods disclosed herein use a robot used to move and/or place sample containers. An imaging device is coupled to the robot and may be used to capture training images of sample containers within diagnostic laboratory system. The use of the robot enables specific movements between the imaging device and the sample containers so the images of the sample containers may include predetermined variations between the images. The variations between the images may include different poses, illumination intensities, illumination spectrums, exposure times, and other imaging conditions. Thus, numerous and varying training images may be obtained within a deployed diagnostic laboratory system.

Further, in some embodiments, once images are annotated, the annotated images may be used to retrain how future images are annotated. That is, a first set of images taken under a first set of conditions (e.g., illumination, pose, motion profile, etc.) may be annotated and then used to train how the AI model of the laboratory system annotates a subsequent, second set of captured images taken under a different, second set of conditions (e.g., different illumination, pose, motion profile, and/or the like). For example, AI used to annotate images, referred to herein as an “annotation generator” for convenience, may be trained to annotate well-lit sample containers imaged using the controllably movable imaging device (e.g., the imaging device affixed to the robot). Once the annotation generator is trained with the well-lit sample container images, a first set of images of a tray of sample containers may be acquired under the same, well-lit condition and the annotation generator may annotate the first set of images. Because the annotation generator was trained using images under the well-lit condition, the annotation of the first set of images should be accurate. Thereafter, the lighting condition may be reduced (e.g., to half intensity or another reduced intensity), and a second set of images may be captured by the imaging device. The precise control provided by the robot allows the imaging device to be positioned in exactly the same viewing position to take a second set of images on exactly the same tray of sample containers. Because all conditions except lighting intensity were the same during capture of the first and second images sets, the annotation used for the first set of images may serve as the annotation for the second set of images. With the annotation and second set of images, the annotation generator may be refined (e.g., retrained) to annotate images taken under the reduced-lighting condition (e.g., half intensity). As such, the annotation generator itself may be part of the sample characterization algorithm and may be trained iteratively to handle more and more variations. That is, controllable movement of the imaging device allows the annotation of a first set of images taken under a first set of conditions to be used to annotate a second set of images take under a second set of conditions (e.g., different illumination intensity, different illumination spectrum, different motion profile, different sample container position, or the like). The above process may be employed to train the annotation generator to annotate images of sample containers, sample container holders, or other image features, using a wide variation of imaging conditions within an actual deployed diagnostic laboratory system.

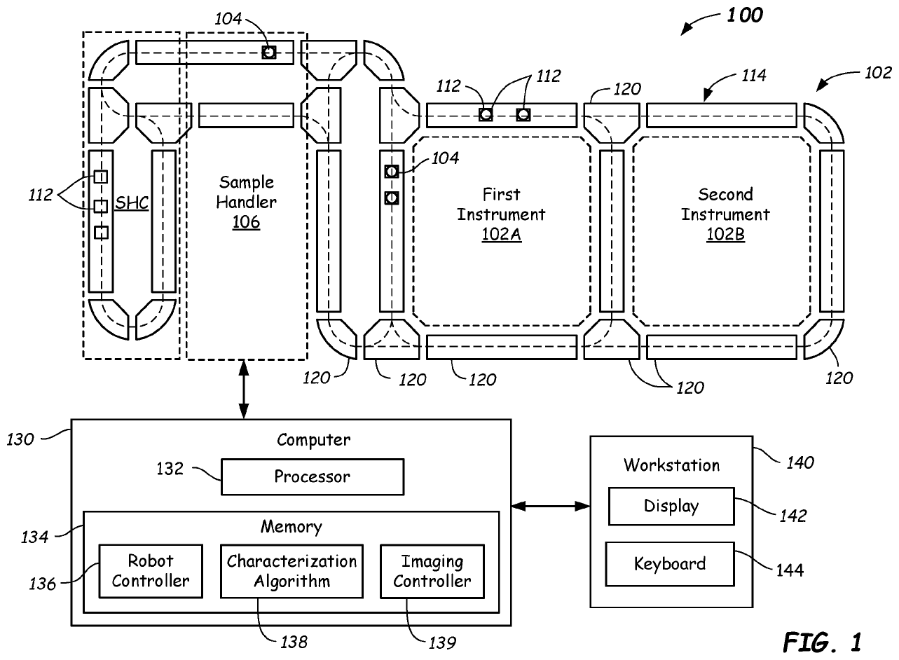

These and other systems and methods are described below in greater detail with reference to FIGS. 1-13F.

Reference is now made to FIG. 1, which illustrates a block diagram of an example embodiment of a diagnostic laboratory system 100. The laboratory system 100 may include a plurality of instruments 102 configured to process the sample containers 104 (a few labelled) and to conduct assays or tests on samples located in the sample containers 104. The laboratory system 100 may have a first instrument 102A and a second instrument 102B. Other embodiments of the laboratory system 100 may include more or fewer instruments.

The samples located in the sample containers 104 may be various biological specimens collected from individuals, such as patients being evaluated by medical professionals. The samples may be collected from the patients and placed directly into the sample containers 104. The sample containers 104 may then be delivered to the laboratory system 100. As described in greater detail below, the sample containers 104 may be loaded into a sample handler 106, which may be an instrument of the laboratory system 100. From the sample handler 106, the sample containers 104 may be transferred into sample carriers 112 (a few labelled) that transport the sample containers 104 throughout the laboratory system 100, such as to the instruments 102, by way of a track 114.

The track 114 is configured to enable the sample carriers 112 to move throughout the laboratory system 100 including to and from the sample handler 106. For example, the track 114 may extend proximate or around at least some of the instruments 102 and the sample handler 106 as shown in FIG. 1. The instruments 102 and the sample handler 106 may have devices, such as robots (not shown in FIG. 1), that transfer the sample containers 104 to and from the sample carriers 112. The track 114 may include a plurality of segments 120 (a few labelled) that may be interconnected. In some embodiments, some of the segments 120 may be integral with one or more of the instruments 102.

Components, such as the sample handler 106 and the instruments 102, of the laboratory system 100 may include or be coupled to a computer 130 configured to execute one or more programs that control the laboratory system 100 including components of the sample handler 106. The computer 130 may be configured to communicate with the instruments 102, the sample handler 106, and other components of the laboratory system 100. The computer 130 may include a processor 132 configured to execute programs including programs other than those described herein. The programs may be implemented in computer code.

The computer 130 may include or have access to memory 134 that may store one or more programs and/or data described herein. The memory 134 and/or programs stored therein may be referred to as non-transitory computer-readable mediums. The programs may be computer code executable on or by the processor 132. The memory 134 may include a robot controller 136 (e.g., computer code executable by processor 132) configured to generate instructions to control robots and/or similar devices in the instruments 102 and the sample handler 106. As described herein, the instructions generated by the robot controller 136 may be in response to data, such as image data received from the sample handler 106.

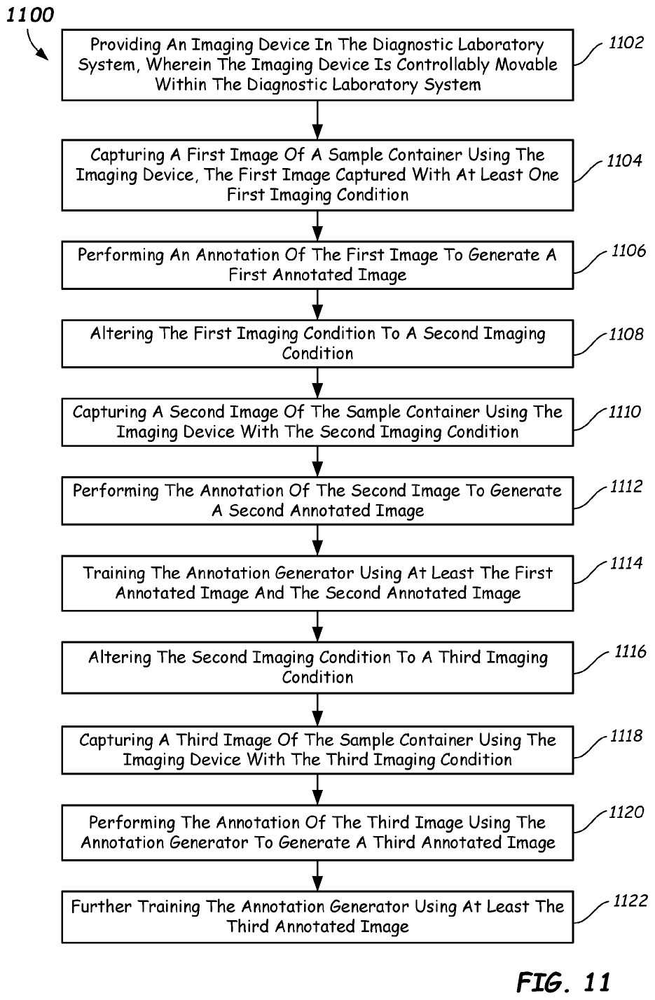

The memory 134 may also store a sample characterization algorithm 138 (e.g., a classification algorithm or other suitable computer code) that is configured to identify and/or classify the sample containers 104 and/or other items in the sample handler 106. In some embodiments, the characterization algorithm 138 classifies objects in image data generated by imaging devices described herein. The characterization algorithm 138 may include a trained model, such as one or more neural networks. In some embodiments, the characterization algorithm 138 may include an annotation generator (912-FIG. 9) configured to annotate images captured by the imaging devices. The characterization algorithm 138 also may include a convolutional neural network (CNN) trained to characterize or identify objects in image data. The trained model is implemented using artificial intelligence (AI). Thus, the trained model may learn to classify sample containers 104 as described herein. It is noted that the characterization algorithm 138 is not a lookup table but rather a supervised or unsupervised model that is trained to characterize and/or identify various types of the sample containers 104.

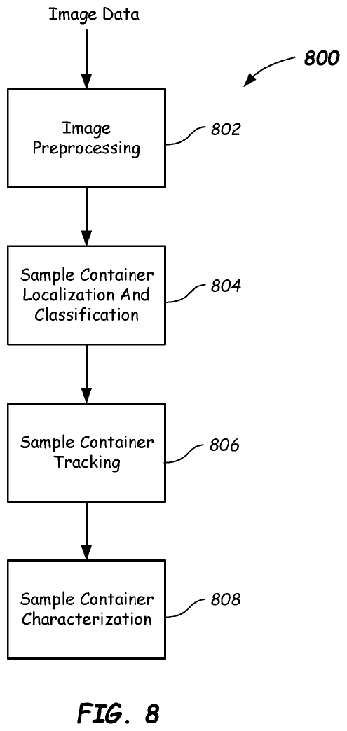

The characterization algorithm 138 may also include one or more algorithms that train AI (e.g., neural networks or other AI models) used to annotate, classify, and/or identify the sample containers 104. The AI may be trained based on training images captured by at least one imaging device (not shown in FIG. 1, see cameras 636, 638 of FIG. 6, for example). In some embodiments, the training images may be captured within the sample handler 106. There may be relative movement between the imaging device and the sample containers. For example, a robot located in one or more of the instruments 102 and/or the sample handler 106 may be configured to move the imaging device relative to the sample containers 104. Additionally, the robot may be configured to move the sample containers 104 relative to the imaging device. During training, the characterization algorithm 138 may direct the robot controller 136 to generate instructions to move the robot to specific locations to capture specific images of the sample containers 104.

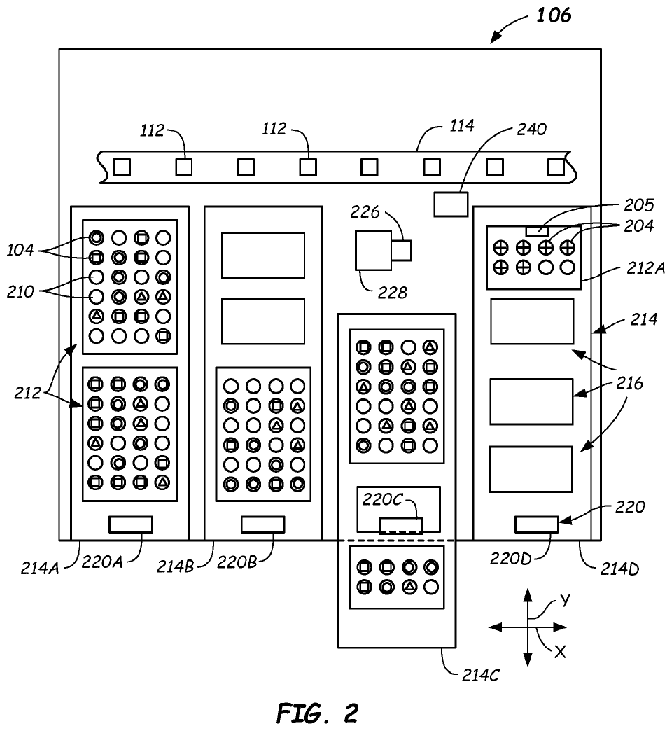

An imaging controller 139 may be implemented in the computer 130. For example, the imaging controller 139 may be computer code stored in the memory 134 and executed by the processor 132. The imaging controller 139 may be configured to control imaging devices (e.g., imaging devices 226, 240—FIG. 2) and illumination sources (e.g., illumination sources 642, 652—FIG. 6) during image capturing. For example, the imaging controller 139 may control cameras (e.g., cameras 636, 638—FIG. 6), such as by setting predetermined frame rates and exposure times during imaging. The imaging controller 139 may also set the illumination intensity and spectrum of light used to illuminate the sample containers 104 during imaging.

The computer 130 may be coupled to a workstation 140 configured to enable users to interface with the laboratory system 100. The workstation 140 may include a display 142, a keyboard 144, and other peripherals (not shown). Data generated by the computer 130 may be displayable on the display 142. In some embodiments, the data may include warnings of anomalies detected by the characterization algorithm 138. The anomalies may include notices that certain ones of the sample containers 104 cannot be characterized. In addition, a user may enter data into the computer 130 by way of the workstation 140. For example, the data entered by the user may be instructions that cause the robot controller 136, the characterization algorithm 138, or the imaging controller 139 to perform certain operations such as capturing and/or analyzing images of sample containers 104. Other data entered by a user may include annotation of training images used during training of the characterization algorithm 138.

Additional reference is now made to FIG. 2, which illustrates a top plan view of the interior of the sample handler 106 according to one or more embodiments. The sample handler 106 is configured to capture images of the sample containers 104 and to transport the sample containers 104 between holding locations 210 (a few labelled) and the sample carriers 112. In the embodiment of FIG. 2, the holding locations 210 are located within trays 212 that may be removable from the sample handler 106. The sample handler 106 may include a plurality of slides 214 that are configured to hold the trays 212. In some embodiments, the sample handler 106 may include four slides 214 that are referred to individually as a first slide 214A, a second slide 214B, a third slide 214C, and a fourth slide 214D. The third slide 214C is shown partially removed from the sample handler 106, which may occur during replacement of trays 212. Other embodiments of the sample handler 106 may include fewer or more slides than are shown in FIG. 2.

Each of the slides 214 may be configured to hold one or more trays 212. In the embodiment of FIG. 2, the slides 214 may include receivers 216 that are configured to receive the trays 212. Each of the trays 212 may contain a plurality of holding locations 210, wherein each of the holding locations 210 may be configured to receive one of the sample containers 104. In the embodiment of FIG. 2, the trays may vary in size to include large trays with twenty-four holding locations 210 and small trays with eight holding locations 210. Other configurations of the trays 212 may include different numbers of holding locations 210 and holding locations configured to hold more than one sample container.

In some embodiments, the sample handler 106 may include one or more slide sensors 220 that are configured to sense movement of one or more of the slides 214. The slide sensors 220 may generate signals indicative of slide movement, wherein the signals may be received and/or processed by the robot controller 136 as described herein. In the embodiment of FIG. 2, the sample handler 106 includes four slide sensors 220 arranged so that each of the slides 214 is associated with one of the slide sensors 220. A first slide sensor 220A senses movement of the first slide 214A, a second slide sensor 220B senses movement of the second slide 214B, a third slide sensor 220C senses movement of the third slide 214C, and a fourth slide sensor 220D senses movement of the fourth slide 214D. Various techniques may be employed by the slide sensors 220 to sense movement of the slides 214. In some embodiments, the slide sensors 220 may include mechanical switches that toggle when the slides 214 are moved wherein the toggling generates an electrical signal indicating that a slide has moved. In other embodiments, the slide sensors 220 may include optical sensors that generate electrical signals in response to movement of the slides 214. In yet other embodiments, the slide sensors 220 may be imaging devices that generate image data of the sample containers 104 as the slides 214 move.

The sample handler 106 may receive many different types of sample containers 104. A first type of the sample containers 104 are noted by triangles, a second type of the sample containers 104 are noted by squares, and a third type of the sample containers 104 are noted by circles. The characterization algorithm 138 is configured to classify the sample containers 104 so that the sample containers 104 may be readily identified by the computer 130 (FIG. 1). The characterization algorithm 138 may also characterize new types of sample containers (e.g., sample containers 204) as described herein.

The sample handler 106 includes sample containers 204 (marked as crosses) that are of a new type or that have not been classified by the characterization algorithm 138. In the embodiment of FIG. 2, the sample containers 204 are placed into a tray 212A that may be designated to hold new types of sample containers. For example, when the sample containers 204 are determined to be in the tray 212A, the computer 130 may determine whether the sample containers 204 are of a new type. If the sample containers 204 are of a new type, the computer 130 may cause the characterization algorithm 138 to classify or characterize the sample containers 204 as described herein.

In some embodiments, the tray 212A may have indicia 205 indicating that the tray 212A contains the new type of sample containers 204. A user may load the sample containers 204 into the tray 212A and insert the tray 212A into the sample handler 106. An imaging device may then capture an image of the indicia 205. The computer 130 may then cause the characterization algorithm 138 to classify the sample containers 204 in response to the detection of the indicia 205. In other embodiments, a user may indicate via the workstation 140 (FIG. 1) that the sample containers 204 have been received in the sample handler 106. In some embodiments, the user may indicate the locations of the sample containers 204 in the sample handler 106.

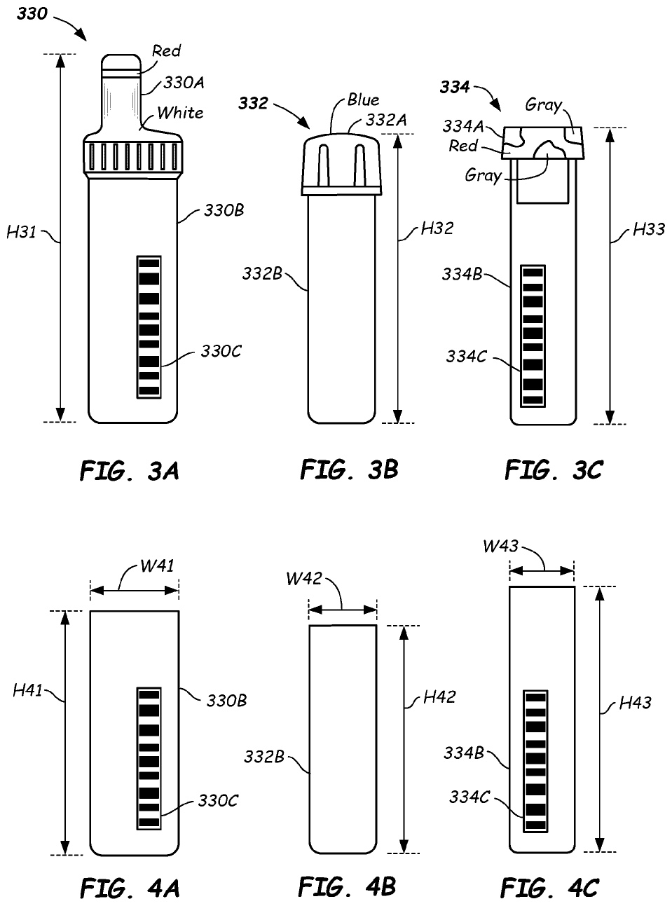

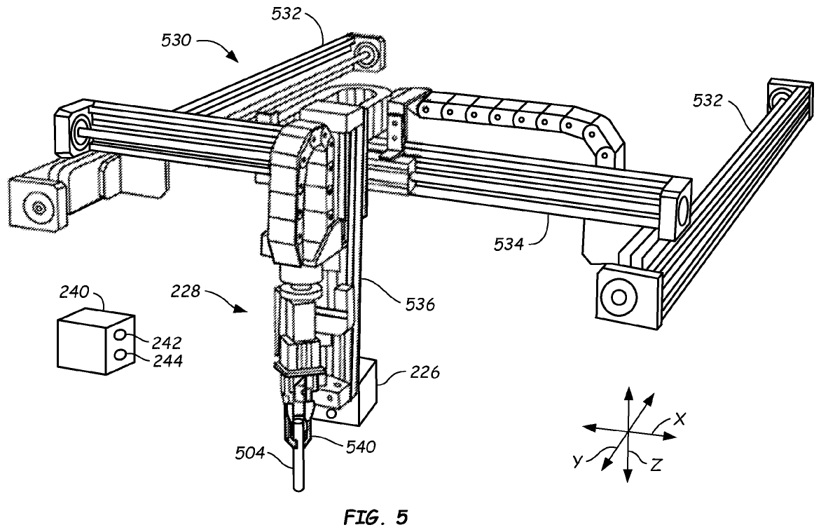

Additional reference is now made to FIGS. 3A-3C, which illustrate different types of example sample containers that may be used within the laboratory system 100. Other types of sample containers may be employed. In some embodiments, sample containers include tubes with or without caps attached to the tubes. Sample containers may also include samples or other contents (e.g., liquids) located in the sample containers. Additional reference is also made to FIGS. 4A-4C, which illustrate the sample containers of FIGS. 3A-3C without the caps. As shown in the figures, all the sample containers may have different configurations or geometries. For example, the caps and the tubes of the different sample container types may each have different features, such as different tube and cap geometries and/or colors. The unique features of the sample containers may be classified and identified by the characterization algorithm 138 (FIG. 1) as described herein. The features described herein also may be used to train the characterization algorithm 138 (as described below).

An example sample container 330 of FIG. 3A includes a cap 330A that is white with a red stripe and has an extended vertical portion. The cap 330A fits over a tube 330B. The sample container 330 has a height H31. FIG. 4A illustrates the tube 330B without the cap 330A. The tube 330B has a tube geometry including a height H41 and a width W41. The tube 330B may have a tube color, a tube material, and/or a tube surface property (e.g., reflectivity). These dimensions, ratios of dimensions, and other properties may be referred to as features and may be used during classification by the characterization algorithm 138 to classify and/or identify the sample container 330.

An example sample container 332 of FIG. 3B includes a cap 332A that is blue with a dome-shaped top and fits over a tube 332B. The sample container 332 has a height H32. FIG. 4B illustrates the tube 332B without the cap 332A. The tube 332B may have tube geometry including a height H42 and a width W42. The tube 332B also may have a tube color, a tube material, and/or a tube surface property. These dimensions, ratios of dimensions, and other properties may be referred to as features and may be used during classification by the characterization algorithm 138 to classify and/or identify the sample container 332.

An example sample container 334 of FIG. 3C includes a cap 334A that is red and gray with a flat top and fits over a tube 334B. The sample container 334 has a height H33. FIG. 4C illustrates the tube 334B without the cap 334A. The tube 334B also may have a tube geometry including a height H43 and a width W43. The tube 334B may have a tube color, a tube material, and/or a tube surface property. These dimensions, ratios of dimensions, and other properties may be referred to as features and may be used during classification by the characterization algorithm 138 to classify and/or identify the sample container 332.

The tube 330B has identifying indicia in the form of a barcode 330C and the tube 334B has identifying indicia in the form of a barcode 334C. Images of the barcode 330C and the barcode 334C may be analyzed by the characterization algorithm 138 for classification purposes as described herein. The barcodes may be referred to as features and may be used to train the characterization algorithm 138 (as described below).

Different types of sample containers may have different characteristics, such as different sizes, different surface properties, and different chemical additives therein as shown by the sample containers 330, 332, and 334 of FIGS. 3A-3C. For example, some sample container types are chemically active, meaning the sample containers contain one or more additive chemicals that are used to change or retain a state of the samples stored therein or otherwise assist in sample processing by the instruments 102. In some embodiments, the inside wall of a tube may be coated with the one or more additives or additives may be provided elsewhere in the sample container. In some embodiments, the types of additives contained in the tubes may be serum separators, coagulants such as thrombin, anticoagulants such as EDTA or sodium citrate, anti-glycosis additives, or other additives for changing or retaining a characteristic of the samples. For example, the sample container manufacturers may associate the colors of the caps on the tubes and/or shapes of the tubes or caps with specific types of chemical additives contained in the sample containers.

Different manufacturers may have their own standards for associating attributes of the sample containers, such as cap color, cap shape (e.g., cap geometry), and tube shape with particular properties of the sample containers. For example, the attributes may be related to the contents of the sample containers or possibly whether the sample containers are provided with vacuum capability. In some embodiments, a manufacturer may associate all sample containers with gray colored caps with tubes including potassium oxalate and sodium fluorate configured to test glucose and lactate. Sample containers with green colored caps may include heparin for stat electrolytes such as sodium, potassium, chloride, and bicarbonate. Sample containers with lavender caps may identify tubes containing EDTA (ethylenediaminetetraacetic acid-an anticoagulant) configured to test CBC with differential., HgBA1c, and parathyroid hormone. Other cap colors such as red, yellow, light blue, royal blue, pink, orange, and black may be used to signify other additives or lack of an additive. In other embodiments, combinations of colors of the caps may be used, such as yellow and lavender to indicate a combination of EDTA and a gel separator, or green and yellow to indicate lithium heparin and a gel separator.

The laboratory system 100 may use the sample container attributes for further processing of the sample containers 104 and/or the samples contained in the sample containers 104. Since the sample containers 104 may be chemically active and affect tests on the samples stored therein, it is important to associate specific tests that can be performed on samples with specific sample container types. Thus, the laboratory system 100 may confirm that tests being run on samples in the sample containers 104 are correct by analyzing the colors and/or shapes of the caps and/or the tubes. Other container attributes may also be analyzed.

Referring again to FIG. 2, the sample handler 106 may include an imaging device 226 that is movable throughout the sample handler 106. In the embodiment of FIG. 2, the imaging device 226 is affixed to a robot 228 that is movable along an x-axis (e.g., in an x-direction) and a y-axis (e.g., in a y-direction) throughout the sample handler 106. In some embodiments, the imaging device 226 may be integral with the robot 228. In one or more embodiments, the robot 228 additionally may be movable along a z-axis (e.g., in a z-direction), which is into and out of the page. In other embodiments, the robot 228 may include one or more components (not shown in FIG. 2) that move the imaging device 226 in the z-direction.

In some embodiments, the robot 228 may receive movement instructions generated by the robot controller 136 (FIG. 1). The instructions may be data indicating x, y, and z positions that the robot 228 should move to. In other embodiments, the instructions may be electrical signals that cause the robot 228 to move in the x-direction, the y-direction, and the z-direction. The robot controller 136 may generate the instructions to move the robot 228 in response to one or more of the slide sensors 220 detecting movement of one or more of the slides 214, for example. The instructions may cause the robot 228 to move while the imaging device 226 captures images of newly-added sample containers 204.

The imaging device 226 includes one or more cameras (not shown in FIG. 2; see cameras 636, 638 of FIG. 6, for example) that capture images, wherein capturing images generates image data representative of the images. The image data may be transmitted to the computer 130 to be processed by the characterization algorithm 138 as described herein. The one or more cameras are configured to capture images of the sample containers 104, 204 and/or other locations or objects in the sample handler 106. The images may be tops and/or sides of the sample containers 104, 204, for example. In some embodiments, the robot 228 may be a gripper robot that grips the sample containers 104, 204 and transfers the sample containers 104, 204 between the holding locations 210 and the sample carriers 112. In such embodiments, the images may be captured while the robot 228 is gripping the sample containers 104, 204 as described herein.

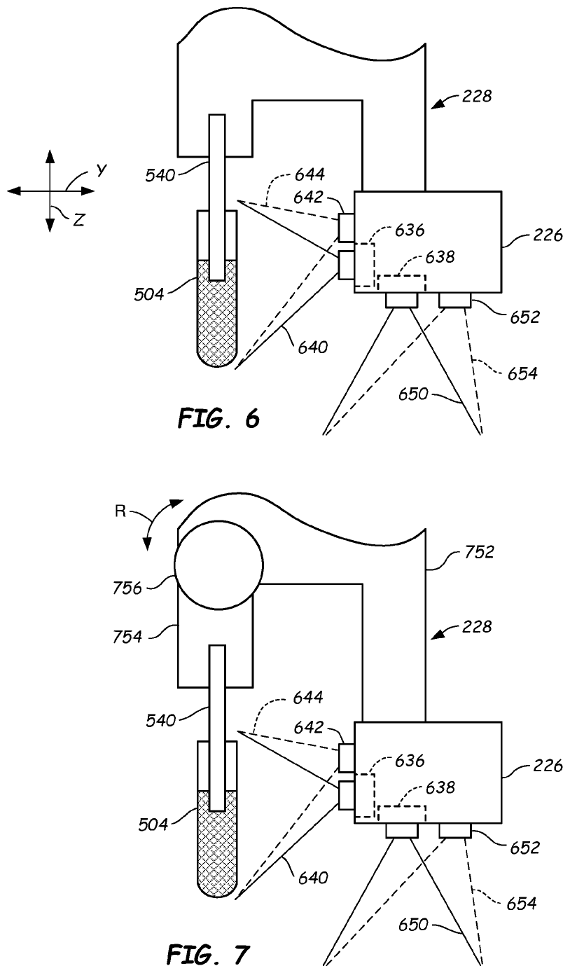

Additional reference is made to FIG. 5, which is a perspective view of an embodiment of the robot 228 coupled to a gantry 530 that is configured to move the robot 228 in the x-direction, the y-direction, and the z-direction. The gantry 530 may include two y-slides 532 that enable the robot 228 to move in the y-direction, an x-slide 534 that enables the robot 228 to move in the x-direction, and a z-slide 536 that enables the robot 228 to move in the z-direction. In some embodiments, movement in the three directions may be simultaneous and may be controlled by instructions generated by the robot controller 136 (FIG. 1). For example, the robot controller 136 may generate instructions that cause motors (not shown) coupled to the gantry 530 to move the slides in order to move the robot 228 and the imaging device 226 to predetermined locations or in predetermined directions.

In some embodiments, the robot 228 may include a gripper 540 (e.g., end effector) configured to grip a sample container 504. The sample container 504 may be an example of one of the sample containers 104 or one of the sample containers described in FIGS. 3A-3C. The robot 228 is moved to a position above a holding location and then moved in the z-direction to retrieve the sample container 504 from the holding location. The gripper 540 opens and the robot 228 moves down in the z-direction so that the gripper 540 extends over the sample container 504. The gripper 540 closes to grip the sample container 504 and the robot 228 moves up in the z-direction to extract the sample container 504 from the holding location. As shown in FIG. 5, the imaging device 226 may be affixed to the robot 228, so the imaging device 226 may move with the robot 228 and capture images of the sample container 504 and other sample containers 104, 204 (FIG. 2) located in the sample handler 106. The imaging device 226 includes at least one camera configured to capture images, wherein the captured images are converted to image data for processing such as by the characterization algorithm 138. The image data may be used to train the characterization algorithm 138. In some embodiments, the image data may train or update training of the annotation generator 912 (FIG. 9).

Additional reference is made to FIG. 6, which is a side elevation view of an embodiment of the robot 228 gripping the sample container 504 with the gripper 540 while the sample container 504 is being imaged by the imaging device 226. The imaging device 226 depicted in FIG. 6 may include a first camera 636 and a second camera 638. Other embodiments of the imaging device 226 may include a single camera or more than two cameras. The first camera 636 has a field of view 640 extending at least partially in the y-direction and may be configured to capture images of the sample container 504 being gripped by the gripper 540. A first illumination source 642 may illuminate the sample container 504 in the field of view 640 by way of an illumination field 644. In some embodiments, the spectrum and/or intensity of light emitted by the first illumination source 642 may be controlled by the characterization algorithm 138 (FIG. 1) and/or the imaging controller 139 (FIG. 1). In other embodiments, the imaging controller 139 is configured to control at least one of intensity of the first illumination source 642 and a spectrum of light emitted by the first illumination source 642.

The second camera 638 may have a field of view 650 that extends in the z-direction and may capture images of the trays 212, the sample containers 104, 204 located in the trays 212, and other objects in the sample handler 106. A second illumination source 652 may illuminate objects in the field of view 650 by an illumination field 654. In some embodiments, the spectrum and/or intensity of light emitted by the second illumination source 652 may be controlled by the imaging controller 139. The field of view 650 and the illumination field 654 enables images of the tops (e.g., caps) of the sample containers 104, 204 to be captured as shown in FIG. 2. The captured images may be analyzed by the characterization algorithm 138 (FIG. 1) to classify or identify the sample containers 104, 204 and/or to determine whether any anomalies are present in the sample handler 106. In some embodiments, the imaging device 226 may have a single camera with a field of view that may capture at least a portion of the sample handler 106 and one or more of the holding locations 210 with or without the sample containers 104, 204 located therein.

In some embodiments, images may be captured as the robot 228 moves the imaging device 226 relative to the sample containers 104, 204. The robot controller 136 (FIG. 1) may set the velocity and direction of the robot 228 relative to the sample containers 104, 204 during image capture.

Operation of the first camera 636, the second camera 638, the first illumination source 642, and/or the second illumination source 652 may be controlled by the imaging controller 139 (FIG. 1). The imaging controller 139 may set one or imaging conditions for these devices during imaging as described herein. For example, the imaging controller 139 may set exposure time, frame rate, illumination intensity, and/or illumination spectrum during image capture. In some embodiments, the characterization algorithm 138 may determine the imaging conditions. Further images may be captured under second imaging conditions or altered imaging conditions.

The images captured by the imaging device 226 may be analyzed by the characterization algorithm 138 to determine characteristics of the sample container 504, the robot 228, the sample containers 104, 204, and other components in the sample handler 106 as described herein. For example, the characterization algorithm 138 may characterize or identify the container type for the sample containers 104, 204, 504. When image data generated by the first camera 636 is analyzed, the characterization algorithm 138 may analyze side views of the sample container 504. The characterization algorithm 138 may also determine whether the sample container 504 is being properly gripped by the gripper 540. When image data generated by the second camera 638 is analyzed, the tops or caps of the sample containers 104, 204 may be characterized. Images generated during the different views may also be used to train or update training of the annotation generator 912 (as described below with reference to FIG. 9).

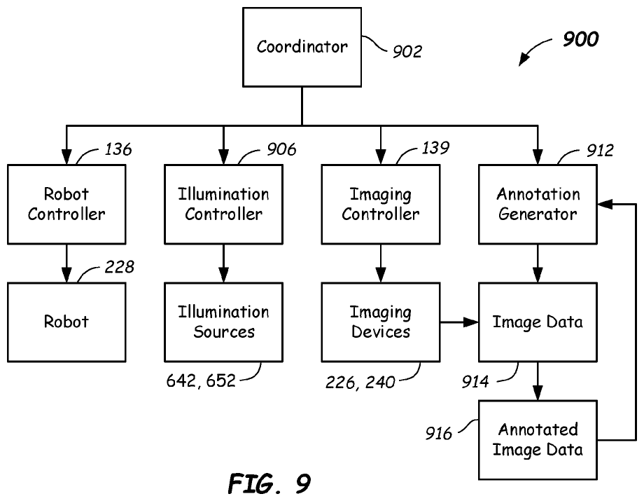

Additional reference is made to FIG. 7, which is a side elevation view of another embodiment of the robot 228 of FIG. 6 with the gripper 540 pivotally coupled to a main structure 752 of the robot 228. This embodiment of the robot 228 includes a secondary arm 754 coupled to the main structure 752 by a pivot mechanism 756, which enables the secondary arm 754 to rotate in an arc R relative to the main structure 752. In the embodiment of FIG. 7, the gripper 540 is coupled to the secondary arm 754 and the imaging device 226 is coupled to the main structure 752, so the sample container 504 may pivot relative to the imaging device 226, which enables images of the sample container 504 in different poses, such as tilted, when captured. In some embodiments, the pivot mechanism 756 enables the secondary arm 754 to pivot in directions other than the arc R, such as directions that are into and out of the paper. The characterization algorithm 138 may determine the poses of the sample container 504 relative to the imaging device 226 and the robot controller 136 may generate instructions to move the robot 228 into the correct poses. Images generated during the different poses may be used to train or update training of the annotation generator 912 (FIG. 9).

Referring again to FIGS. 2 and 5, in some embodiments, the sample handler 106 includes a fixed imaging device 240 that may be in a fixed location. In such embodiments, the robot 228 may move the sample containers 104, 204 proximate the imaging device 240 where the imaging device 240 may then capture images of the sample containers 104, 204. The images generated by the imaging device 240 may be processed as described herein, such as by the characterization algorithm 138. The imaging device 240 may include a camera 242 and an illumination source 244, wherein the illumination source 244 may be configured to illuminate objects being imaged by the camera 242. In some embodiments, the spectrum and/or intensity of light emitted by the illumination source 244 may be controlled by the characterization algorithm 138 and/or the imaging controller 139.

Having described example embodiments of the laboratory system 100, methods of processing image data generated by the laboratory system 100 will now be described. The laboratory system 100 and methods described herein generate data and annotations with real world variations by using the combination of the imaging device 226 and the robot 228 to generate image data. Embodiments are applied to sample container characterization wherein the characterization may include characterizing sample containers 104, 204 and/or 504 with and without caps and samples contained in the sample containers 104, 204 and/or 504. The characterization may include annotation as described herein.

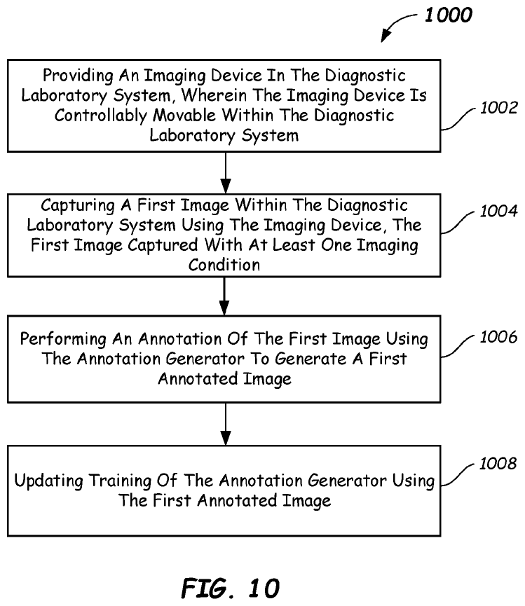

Additional reference is made to FIG. 8, which is a flowchart of an example sample container characterization workflow 800 that may be implemented in the characterization algorithm 138 and executed by processor 132. In some embodiments, the robot 228 may grip the sample container 504 (FIG. 5) and the imaging device 226 may capture images of the sample container 504. In other embodiments, the robot 228 may move the imaging device 226 relative to the sample containers 104, 204 so that the imaging device 226 may capture images of specific ones of the sample containers 104, 204. During image capture, respective ones of the illumination sources 642 or 652 (FIGS. 6 and 7) may illuminate the sample containers 104, 204, 504 using predetermined intensities and spectrums of light (e.g., full intensity, half intensity, and the like, white light, red light, green light, blue light, and the like, etc.). The predetermined intensities and spectrums of light may be determined by the characterization algorithm 138 in some embodiments. The imaging device 226 may then capture images of the sample containers 104, 204, 504 under these illumination conditions. The imaging controller 139 may set other illumination conditions during image capture. The images may be captured using one or both of the first camera 636 and the second camera 638 in the imaging device 226. Thus, the images may include the tops of the sample containers 104, 204 and/or the sample container 504 being gripped by the gripper 540. The sample container 504 may have different poses relative to the imaging device 226 (e.g., using pivot mechanism 756).

The image data may be received at operational block 802 where preprocessing such as deblur, gamma correction, and radial distortion correction may be performed before further processing. A data-driven machine-learning approach, such as a generative adversarial network (GAN) or another suitable AI network may be used for the preprocessing at operational block 802. Processing may proceed to operational block 804 where the images of the sample container 504 may undergo sample container localization and classification. (The process is described relative to the sample container 504, but is applicable to the sample containers 104, 204.) Localization may be an annotation of images of the sample container 504 to specify the location of sample container 504 within each image and may include surrounding images of the sample container 504 with a virtual box (e.g., a bounding box or a pixelwise mask) to isolate the sample container 504, for example. Classification may be performed using a data-driven machine-learning based approach such as a convolutional neural network (CNN).

The CNN may be enhanced using YOLOv4 or other image identification networks or models. YOLOv4 is a real-time object detection model that works by breaking an object identification task into two operations. The first operation uses regression to identify object positioning via bounding boxes and the second operation uses classification to determine the class of the object (e.g., the sample container 104, 204, or 504). The localization may provide the bounding box for the detected sample container. The classification determines high level characteristics of the sample container such as determining whether the sample container is capped, uncapped, or is a tube top sample cup (TTSC). In some embodiments, the classification also determines a classification confidence.

Processing may proceed to sample container tracking at operational block 806 where, for each newly detected sample container, the computer 130 (e.g., via the robot controller 136 and/or the characterization algorithm 138) may assign a new tracklet identification to each sample container (e.g., an identification of a portion of a path travelled by the sample container such as a portion of track 114). Alternatively, the computer 130 may try to associate a detected sample container with an existing tracklet established in previous images based on an overlapping area between a detected bounding box and a predicted bounding box established on the motion trajectory, classification confidence, and other features derived from the appearance of the image of the sample container. In situations where detections are potentially missed, which prevents tracking, a more sophisticated data association algorithm such as the Hungarian algorithm may be utilized to ensure robustness of the tracking. In some embodiments, deep SORT or other machine-learning algorithms may be used for sample container tracking.

When a tracklet contains sufficient observations collected across multiple images (e.g., frames), the characterization algorithm 138 may start to estimate more detailed characteristics at operational block 808. The characteristics include, but are not limited to, sample container height and sample container diameter, cap color, cap shape, and barcode reading when a bar code or other sample container identification indicia is in a field of view of the imaging device 226 or the imaging device 240.

Training the data-driven machine-learning algorithms, software models, and networks may require collecting image data under varied controlled (e.g., predetermined) conditions. The varied conditions may include different sample container types, lighting conditions (e.g., illumination intensity, illumination spectrum, etc.), camera spectral properties, exposure time, sample container distance and/or pose, relative motions between the imaging device 226 and the sample containers, etc.

FIG. 9 is a diagram of an example workflow 900 for generating image data with varied conditions in accordance with one or more embodiments described herein. With reference to FIG. 9, a coordinator 902 is provided for directing workflow 900. In some embodiments, coordinator 902 may be implemented as computer program code stored in memory 134 (FIG. 1) and executed by processor 132. For example, coordinator 902 may be implemented in the characterization algorithm 138. Coordinator 902 may be coupled to robot controller 136 for controlling robot 228, an illumination controller 906 for controlling operation of illumination sources 642 and 652, imaging controller 139 for controlling operation of imaging devices 226 and 240, and an annotation generator 912 which annotates captured images as described below. Specifically, coordinator 902 may control workflow 900 to generate image data of sample containers by: (1) using robot controller 136 to position robot 228 and imaging device 226 coupled thereto; (2) using illumination controller 906 to illuminate sample containers with illumination source 642 and/or 652 (e.g., using a desired illumination intensity, illumination spectrum, etc.); (3) using imaging controller 139 to direct imaging device 226 and/or 240 to capture images (e.g., image data 914) of the sample containers (e.g., using a desired expose time or other imaging parameters); and (4) using annotation generator 912 to generate annotated captured images (e.g., annotated image data 916). As will be described further below, coordinator 902 may also direct the annotation generator 912 to store the annotation used for one or more images and reuse a stored annotation on one or more subsequently captured images. Additionally, in some embodiments, coordinator 902 may direct retraining of annotation generator 912.

The locations of one or more of the sample containers 104, 204 or 504 that are to be characterized may be stored in at least one of the robot controller 136 or the characterization algorithm 138, for example. In some embodiments, characterization includes annotating or identifying images of the sample containers. For example, the sample containers 204 that are to be characterized may be located in the tray 212A (FIG. 2). The robot controller 136 may generate signals or instructions that cause the robot 228 to retrieve the sample containers 204 and to locate individual ones of the sample containers in specific positions or orientations (e.g., poses) relative to the imaging device 226 (or imaging device 240) so that the imaging device may capture images of the sample containers (e.g., sample containers 204). For example, the specific positions may include predetermined distances from the imaging device 226 (or 240), predetermined poses (e.g., angles) relative to the imaging device 226 (or 240), and relative movement between the sample containers and the imaging device 226 (or 240) during imaging.

Illumination controller 906 manages the illumination intensity and/or spectrum of the illumination sources 642, 652. In some embodiments, the illumination controller 906 may be implemented in the imaging controller 139. The characterization algorithm 138 may generate instructions or imaging requirements that may be translated by the illumination controller 906 to generate the instructions that control the illumination sources 642, 652 and/or the cameras 636, 638. The image controller 139 may direct cameras 636, 638 to generate image data 914, which may be digital data representative of the captured images of the sample containers 204 under the illumination conditions established by the illumination controller 906.

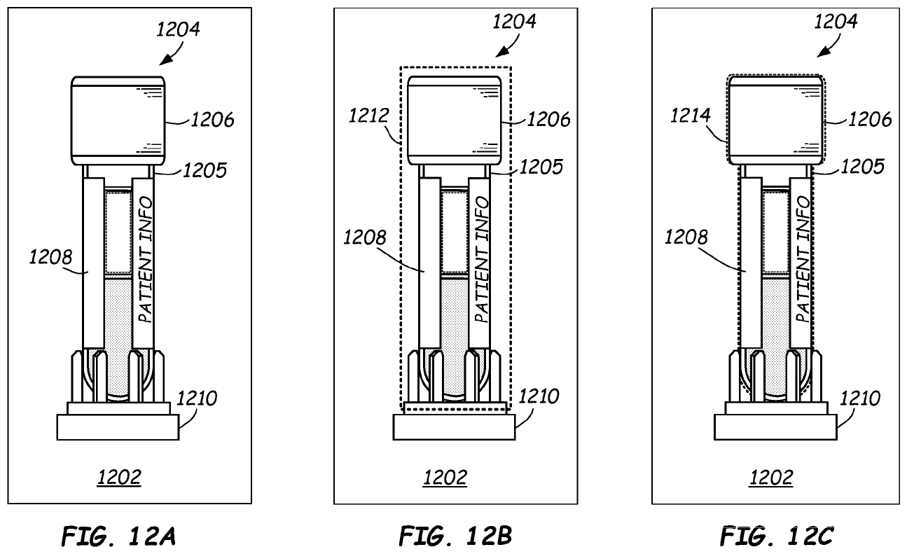

Annotation generator 912 may identify objects in an image and label the objects. During some annotation processes, a bounding box may be generated within the image, wherein the bounding box includes one or more objects that are to be identified. The objects may be identified or classified as classes or instances. For example, annotation generator 912 may be used to identify sample containers as a class of objects in images. In other embodiments, segmentation may be used to identify specific instances, such as the types of sample containers that are in the images. Annotation generator 912 may use tools other than bounding boxes. For example, annotation generator 912 may use polygonal segmentation, semantic segmentation, 3D cuboids, key-points and landmarks, or lines and splines. The annotations then may be used to create training datasets for sample container identification. In some embodiments, annotation generator 912 may include a deep learning network such as a general convolutional neural network (CNN). Example networks include Inception, ResNet, ResNext, DenseNet, or the like, although other CNN and/or AI architectures may be employed. Training of annotation generator 912 is described further below with reference to FIG. 10.

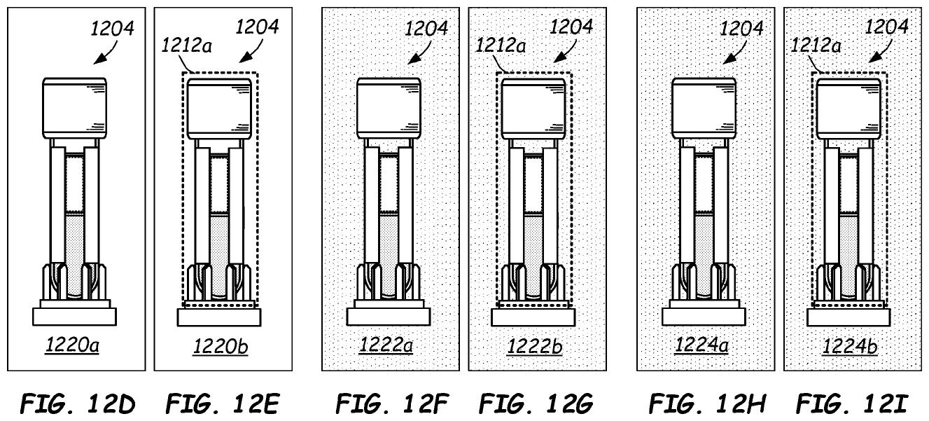

The annotation generator 912 may generate a predicted annotation of the images of the sample containers represented by the image data 914 by leveraging previously annotated data as described further herein. The annotation generator 912 generates annotated image data 916 from image data 914. The annotated image data 916 then may be fed back to the annotation generator 912 for further annotation and/or further training of the annotation generator 912. In some embodiments, a first annotation of one or more objects in a first image may be performed followed by performing a second annotation of the one or more objects in a second image reusing the first annotation on the second image. For example, illumination intensity, illumination spectrum, pose or another condition may be varied between the capture of the first and second images but the annotation of the first image may be used to annotate the second image as described further below with reference to FIG. 11 (e.g., such as if the sample container is expected to be in the same position in both images due to precise positioning of the imaging device 226 by robot 228). The annotation generator 912 may be trained by this process or the annotation generator 912 may have its training updated by this process.

Image annotation as performed by the annotation generator 912 may include the task of annotating images of sample containers with labels. In some embodiments, some annotation may additionally involve human-powered tasks. Labels may be predetermined during programming of the machine learning and are chosen to give the computer vision model (e.g., the characterization algorithm 138) information regarding objects that are in the images. Example considerations during annotation include possible naming and categorization issues, representing occluded objects (e.g., occluded tubes or occluded samples stored in the tubes), labelling parts of the images that are unrecognizable, and other considerations.

Annotating images of the sample containers 104, 204 or 504 by the annotation generator 912 may include applying a plurality of labels to objects in the images of the sample containers by applying bounding boxes to the certain ones of the objects. For example, caps, tubes, and identifying indicia may be bounded by bounding boxes. This process may be repeated and, depending on the required classification, the quantity of labels in each image may vary. Some classifications may require only one label to represent the content of an entire image (e.g., image classification). Other classifications may require multiple objects to be annotated within a single image, each with a different label (e.g., a different bounding box). For example, at least two of a cap, a tube, and identifying indicia may have to be annotated to classify some types of sample containers.

The repeatability of positioning between the robot 228 and the sample containers 104, 204 or 504 enables the above-described methods of annotating objects in the images of the sample containers. For example, an image sequence can be captured with slow movement between the robot 228 and the sample containers 204 under well-lit illumination conditions, which makes annotation relatively easy either in an automated or a semi-supervised way. In addition, by capturing images of the same sample container at multiple known positions or sample container orientations, it is possible to use stereoscope vision or multi-view stereo vision to extract high-resolution depth information of the sample container. The stereo image enables three-dimensional images (3-D images) of the sample containers 104, 204 or 504 to be reconstructed as well as provides detailed features for differentiating sample categories, which may help automate the annotation process. For example, some capped sample containers with a black/gray center and a white outer ring may appear nearly identical to an uncapped sample container when viewed with a single topdown image (e.g., an image captured in the z-direction), such that manual annotation may be required with conventional systems. In some embodiments, the ground truth may be automatically identified based on the 3-D images.

For sample container localization, the annotation may be a bounding box or a binary mask for each sample container in an image. For sample container tracking, the annotation may be a unique identifier for each sample container in a holding location across an image sequence. Then, these annotations may be propagated to another image sequence acquired under different imaging conditions, such as different lighting conditions, motion profiles, viewing positions (e.g., pose), and other imaging conditions in an automated fashion based on the position of the robot 228 and/or the imaging device 226 with respect to the previously annotated image sequence. The annotation generator 912 may be trained on these images such that training is an iterative process.

As described above, the laboratory system 100 may use different types of sample containers 104, 204 or 504, such as from different manufacturers. The laboratory system 100 should know the types of the sample containers 104, 204 or 504 in order to properly transport the sample containers 104, 204 or 504 and process the samples. Robots, such as the robot 228, and the sample carriers 112 may have specific hardware and processes for transporting different types of the sample containers 104, 204 or 504. For example, the robot 228 may grasp a first type of sample container differently than a second type of sample container. In addition, the laboratory system 100 may utilize different types of sample carriers 112 depending on the types of the sample containers. Thus, it is important for the laboratory system 100 to identify the sample containers.

The laboratory system 100 described herein uses vision systems, such as the imaging device 226, to capture images of the sample containers 104, 204 or 504. The characterization algorithm 138 analyzes image data generated by the imaging device 226 (or imaging device 240) to identify and/or classify the sample containers. Other imaging devices may capture images of the sample containers, and the characterization algorithm 138 may analyze the image data generated by these imaging devices.

The characterization algorithm 138 may include AI models that are configured to characterize different types of sample containers and variations of their respective tubes and/or the caps. As new types of sample containers are introduced into the laboratory system 100, the AI models in the characterization algorithm 138 should be updated to be able to classify the new types of sample containers. As previously described, retraining the AI models in conventional laboratory systems may be costly and time consuming. The laboratory system 100 described herein overcomes the problems with new sample container classification by training the annotation generator 912 as described herein.

In another example, the laboratory system 100 may receive a new sample container type from a manufacturer. In some embodiments, the new type of sample containers may be loaded into the tray 212A (FIG. 2). Each attribute of the new sample containers may be similar to the attributes of a specific sample container on which the characterization algorithm 138 (e.g., including the annotation generator 912) has been trained. For example, the new sample containers 204 may have the same tube material as a sample container on which the characterization algorithm 138 has been trained but with a different cap shape. Another sample container type on which the characterization algorithm 138 has been trained may have the same cap type as the new sample container type, but with a different tube material. The characterization algorithm 138 (and annotation generator 912) may be trained on the new sample containers (e.g., by employing one or more annotations from previous images to annotate images of the new sample container type and then retraining the annotation generator 912).

In some embodiments, a user may receive a new type of sample container or a sample container that had not been properly identified and load the sample container into one of the trays 212, such as the tray 212A (FIG. 2). For example, the user may slide the tray 212A into the sample handler 106 via the fourth slide 214D. As the fourth slide 214D is slid into the sample handler 106, the fourth slide sensor 220D may detect the movement and may capture an image of the indicia 205, which may indicate that the tray 212A contains the new sample container. In other embodiments, the user may input data via the workstation 140 (FIG. 1) indicating that the new sample container is located in the tray 212A. In some embodiments, the tray 212A may also contain similar sample containers that may be imaged and used to train the annotation generator 912.

The characterization algorithm 138, through use of the coordinator 902 (FIG. 9), may transmit instructions to the robot controller 136 that cause the robot 228 to move to predetermined positions so the imaging device 226 can capture images of the sample containers 104, 204 or 504. The images may be captured by one or both of the first camera 636 and the second camera 638 and under different imaging conditions. For example, the images may be captured under different illumination and camera conditions as determined by the characterization algorithm 138 and the imaging controller 139.

In some embodiments, the robot 228 may grasp a sample container and extract the sample container from the tray 212A as shown in FIG. 5. The imaging device 226 may then capture images of the sample container. In some embodiments, the robot 228 may return the sample container to the tray 212A and re-grasp the sample container so the sample container is at a different orientation relative to the imaging device 226. The imaging device 226 may then capture new images for processing as described herein. Referring to FIG. 7, the robot 228, via the secondary arm 754 may rotate a sample container relative to the imaging device 226. The imaging device 226 may then capture images of the sample container at the different orientations. In some embodiments, the imaging device 226 may capture images of the sample container as the sample container is rotated relative to the imaging device 226. The rate of rotation may be one of the imaging conditions described herein.

The second camera 638 (FIG. 6) may capture images from the tops of the sample containers similar to the methods described above with regard to the first camera 636. In some embodiments, the robot may move the imaging device 226 relative to the sample containers as the second camera 638 captures images of the sample containers. The movement of the imaging device 226 relative to the sample containers may be one of the imaging conditions described herein.

The image data generated by the imaging device 226 may be used to update, train, or retrain the characterization algorithm 138. For example, AI models in the characterization algorithm 138 may be updated using the image data. In some embodiments, the updating or retraining includes training or updating the training of the annotation generator 912 as described below.

Reference is now made to FIG. 10, which illustrates a flowchart of a method 1000 of updating training of an annotation generator (e.g., annotation generator 912) of a diagnostic laboratory system (e.g., laboratory system 100). The method includes, at block 1002, providing an imaging device (e.g., imaging device 226) in the diagnostic laboratory system, wherein the imaging device is controllably movable within the diagnostic laboratory system. The method 1000 includes, at block 1004, capturing a first image within the diagnostic laboratory system using the imaging device, the first image captured with at least one imaging condition (e.g., a predetermined illumination intensity, illumination spectrum, sample container pose, exposure rate, image device and/or sample container speed, etc.). The method 1000 includes, at block 1006, performing an annotation of the first image using the annotation generator to generate a first annotated image. For example, in some embodiments, annotation may include surrounding an image of the sample container with a virtual box or other shape (e.g., a bounding box or a pixel-wise mask) to isolate the sample container.

The method 1000 includes, at block 1008, updating training of the annotation generator using the first annotated image. Annotation generator training may be implemented based on the specific algorithm to be trained. For a sample container detection task, the annotation generator 912 may, for example, annotate a bounding box of the sample container based on the detection algorithm under training. For a sample container classification/identification task, the annotation generator 912 may annotate the class/type of the sample container based on the classification algorithm under training. For a semantic segmentation task, the annotation generator 912 may generate annotation masks on the pixel level for each object region inside the input image. Since annotation occurs on the pixel level, the annotation region can be any irregular shapes (e.g., pixel-level mask, polygons, contours, splines) instead of a predefined bounding box in a shape of a square, rectangle, circle, or oval. In some embodiments, the annotation generator 912 may train one or more tasks at the same time. The annotations generated by the annotation generator 912 may be used with the input image to conduct the next iteration of the algorithm/model training, and the updated algorithm/model may be used by the annotation generator 912 to annotate new input images under different imaging conditions (e.g., different illumination intensity, illumination spectrum, sample container pose, exposure rate, etc.). Then the next iteration of training may be conducted with the annotations and these new input images. In some embodiments, the annotation generator 912 may be implemented through the use of machine-learning approaches so it can learn to process more and more challenging conditions through the iteration of training. For example, the annotation generator 912 may be implemented as a deep neural network algorithm. In some embodiments, annotation generator 912 may include a deep learning network such as a general convolutional neural network (CNN). Example networks include Inception, ResNet, ResNext, DenseNet, or the like, although other CNN and/or AI architectures may be employed. Training may be performed continuously, periodically, or at any suitable time. Training may be performed while the diagnostic laboratory system 100 is online (e.g., in use) or offline.

Reference is now made to FIG. 11, which illustrates a flowchart of another example method 1100 of training an annotation generator (e.g., annotation generator 912) of a diagnostic laboratory system (e.g., laboratory system 100). The method 1100 includes, at block 1102, providing an imaging device (e.g., imaging device 226) in the diagnostic laboratory system, wherein the imaging device is controllably movable within the diagnostic laboratory system. For example, imaging device 226 may be affixed to and move with robot 228. The method 1100 includes, at block 1104, capturing a first image of a sample container (e.g., sample container 104, 204 or 504) using the imaging device, the first image captured with an imaging condition. In one example embodiment, the imaging condition may be illumination intensity. For example, the first image may be captured in a well-lit condition on which the annotation generator 912 had been previously trained. Other example imaging conditions may include illumination intensity, illumination spectrum, sample container and/or imaging device speed, relative position and angle between the imaging device and one or more sample containers, imaging device exposure, imaging device lens properties such as focal length, aperture, and depth of field, etc.

The method 1100 includes, at block 1106, performing an annotation of the first image to generate a first annotated image. For example, the annotation generator 912 may annotate the image using a bounding box, pixel-wise mask, or the like. If the imaging condition used with the first image is an imaging condition on which the annotation generator 912 was previously trained, the annotation provided by the annotation generator 912 should be very accurate.

The method 1100 includes, at block 1108, altering the first imaging condition to a second imaging condition. For example, in the embodiment of the well- lit first image, the imaging condition may be reducing the illumination intensity prior to capturing the second image. Other imaging conditions may be changed. The method 1100 includes, at block 1110, capturing a second image of the sample container using the imaging device with the second imaging condition. The method 1100 includes, at block 1112, performing the annotation of the second image to generate a second annotated image. In some embodiments, the annotation generator 912 may use the same annotation used with the first image. For example, if the imaging condition that is altered is illumination intensity (or illumination spectrum), the precise control provided by the robot 228 allows the imaging device 226 to be positioned in exactly the same viewing position to take a second image on exactly the same tray of sample containers. Because all (or most) conditions except lighting intensity (or spectrum) were the same during capture of the first and second images, the annotation used for the first image may serve as the annotation for the second image. With the annotation and second set of images, the annotation generator may be refined (e.g., retrained) to annotate images taken under the reduced-lighting condition (e.g., half intensity), a different illumination spectrum, or whatever image condition was altered. The method 1100 includes, at block 1114, training the annotation generator using at least the first annotated image and the second annotated image. For example, both the first annotated image and the second annotated image may be included in the training image set used to train the annotation generator 912. Training may be performed continuously, periodically, or at any suitable time.

The method 1100 includes, at block 1116, altering the second imaging condition to a third imaging condition. For example, the imaging condition may include illumination intensity, illumination spectrum, sample container and/or imaging device speed, sample container pose, exposure rate, etc. The method 1100 includes, at block 1118, capturing a third image of the sample container using the imaging device with the third imaging condition. As with the first and second images, in some embodiments, the imaging device 226 may be employed to capture the third image.

The method 1100 includes, at block 1120, performing the annotation of the third image using the annotation generator to generate a third annotated image. In some embodiments, annotator generator 912 may use the same annotation used with the first image or the second image. For example, if the imaging condition that is altered is illumination intensity (or illumination spectrum or exposure rate or the like), the precise control provided by the robot 228 allows the imaging device 226 to be positioned in exactly the same viewing position to take the third image as was used for the first and second images. Likewise, precise changes in position of the imaging device 226 relative to a sample container may be provided between images. In this manner, the annotation used for the first or second image may serve as the annotation for the third image. With the annotation and third image, the annotation generator may be retrained to annotate images taken under the differing imaging conditions such as reduced-lighting, a different illumination spectrum, a different sample container pose, a different exposure rate, or the like.

The method 1100 includes, at block 1122, further training of the annotation generator 912 using at least the third annotated image. As described previously, training may be performed continuously, periodically, or at any suitable time.

With the annotations and the first, second and third images, the annotation generator 912 may be retrained to annotate images taken under differing conditions (e.g., reduced-lighting, different illumination spectrum, different speeds between the sample container and imaging device, different sample container poses, different exposure rates, and the like). As such, the annotation generator 912 itself may be part of the sample characterization algorithm 138 and may be trained iteratively to handle more and more variations. That is, controllable movement of the imaging device 226 allows the annotation of a first set of images taken under a first set of conditions to be used to annotate a second set of images take under a second set of conditions (e.g., different illumination intensity, different illumination spectrum, different motion profile, different sample container position, or the like). This may be extended to a third, fourth, fifth, or other number of image sets and/or imaging conditions. The above process may be employed to train the annotation generator 912 to annotate images of sample containers, sample container holders, or other image features, using a wide variation of imaging conditions within an actual deployed diagnostic laboratory system.

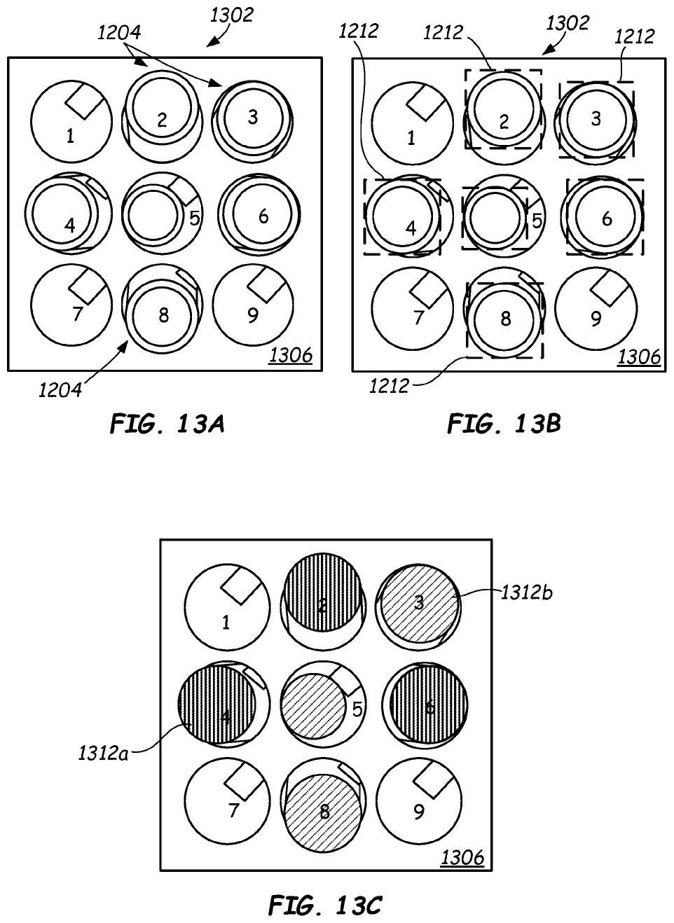

FIGS. 12A-12I illustrate example images and image annotations in accordance with embodiments provided herein. With reference to FIG. 12A, an image 1202 of a sample container 1204 is shown. Sample container 1204 may be similar to previously described sample container 104, 204 or 504 and includes a tube 1205, a cap 1206, and a label 1208. Sample container 1204 is supported on a carrier 1210. FIG. 12B illustrates an example of a bounding box annotation 1212 of sample container 1204 of image 1202, while FIG. 12C illustrates an example of a pixel-wise mask annotation 1214 of sample container 1204. Other annotation types may be employed.

FIG. 12D illustrates an example of a first image 1220a taken under a first imaging condition (e.g., a first illumination intensity). FIG. 12E illustrates an example of a first annotated image 1220b based on the first image 1220a (e.g., using a bounding box 1212a or other suitable annotation). For example, the first imaging condition may be a condition on which annotation generator 912 is trained so that the annotation 1212a of first image 1220a is highly accurate. FIG. 12F illustrates an example of a second image 1222a taken under a second imaging condition different than the first imaging condition used for the first image 1220a. For example, second image 1222a may be taken using a different illumination intensity, illumination spectrum, sample container and/or imaging device speed, relative position and angle between the imaging device 226 and sample container 1204, imaging device exposure, imaging device lens properties such as focal length, aperture, depth of field, or the like (as represented by the light shading in FIGS. 12F and 12G). FIG. 12G illustrates an example of a second annotated image 1222b based on the second image 1222a and using the annotation 1212a of the first annotated image 1220b as previously described. FIG. 12H illustrates an example of a third image 1224a taken under a third imaging condition that is different than either the first or second imaging conditions (as represented by the medium shading in FIG. 12H and 12I). Finally, FIG. 12I illustrates a third annotated image 1224b based on third image 1224a and using the annotation 1212a of either the first annotated image 1220b or the second annotated image 1222b. With the annotated second and/or third images 1222b, 1224b, the annotation generator 912 may be retrained to annotate images taken under the differing imaging conditions such as differing illumination intensity, illumination spectrum, sample container and/or imaging device speed, relative position and angle between the imaging device and one or more sample containers, imaging device exposure, imaging device lens properties such as focal length, aperture, depth of field, or the like.

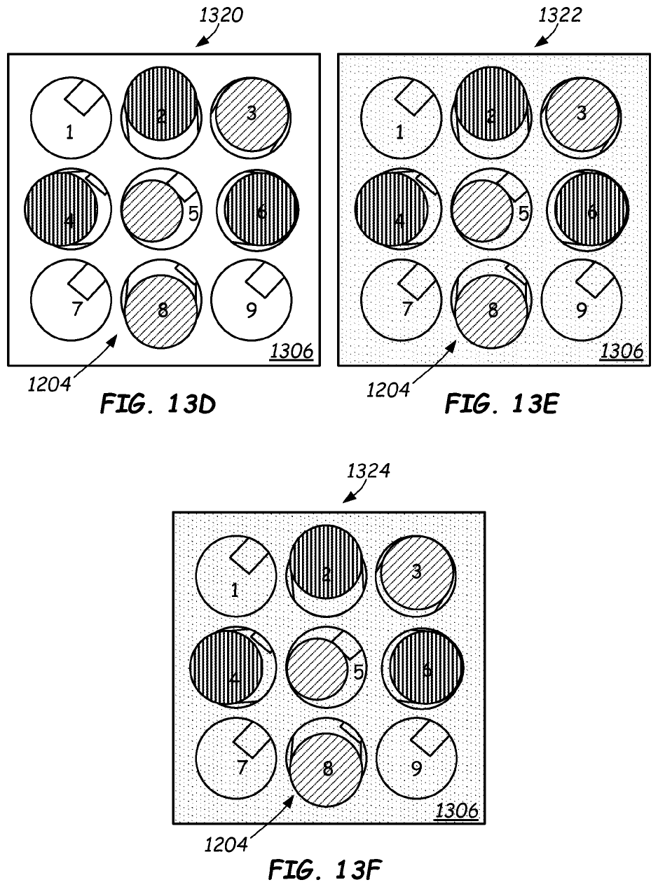

FIGS. 13A-13F illustrate additional example images and image annotations in accordance with embodiments provided herein. With reference to FIG. 13A, an image 1302 of a plurality of sample containers 1204 in a tray 1306 is shown. Sample containers 1204 may be similar to previously described sample containers 104, 204 or 504. FIG. 13B illustrates an example of a bounding box annotation 1212 of sample containers 1204 of image 1302. FIG. 13C illustrates an example of mask annotations 1312a and 1312b of sample containers 1204 which identify different characteristics of the sample containers (e.g., with or without a cap, different cap colors, different cap types, etc.). Other annotation types may be employed.

FIG. 13D illustrates an example of a first annotated image 1320 taken under a first imaging condition (e.g., a first illumination intensity) and using a mask annotation for the top of each sample container 1204. For example, the first imaging condition may be a condition on which annotation generator 912 is trained so that the annotation of first annotated image 1320 is highly accurate. FIG. 13E illustrates an example of a second annotated image 1322 taken under a second imaging condition different than the first imaging condition used for the first annotated image 1320. For example, second annotated image 1322 may be taken using a different illumination intensity, illumination spectrum, sample container and/or imaging device speed, relative position and angle between the imaging device 226 and sample containers 1204, imaging device exposure, imaging device lens properties such as focal length, aperture, depth of field, or the like (as represented by the light shading in FIG. 13E). In some embodiments, second annotated image 1322 may use the annotation of the first annotated image 1320 as previously described. FIG. 13F illustrates an example of a third annotated image 1324 taken under a third imaging condition that is different than either the first or second imaging conditions (as represented by the medium shading in FIG. 13F). In some embodiments, third annotated image 1324 may use the annotation of either the first annotated image 1320 or the second annotated image 1322. With the annotated second and/or third images 1322, 1324, the annotation generator 912 may be retrained to annotate images taken under the differing imaging conditions as described above.

While image capture has been described primarily with regard to imaging device 226, it will be understood that imaging device 240 or any other suitable imaging device may be used.

As described above, annotation generator 912 may be trained to annotate images taken under differing image conditions. Such annotations may allow for more accurate characterization of sample containers and allow for improved substrate handling by substrate handler 106 and/or robot 228. In one or more embodiments, sample containers may be identified using images annotated by annotation generator 912 and robot 228 or another robot may be positioned and/or used to transport sample containers based on images annotated by annotation generator 912.

While the disclosure is susceptible to various modifications and alternative forms, specific method and apparatus embodiments have been shown by way of example in the drawings and are described in detail herein. It should be understood, however, that the particular methods and apparatus disclosed herein are not intended to limit the disclosure.

What is claimed is:

1. A method of updating training of a sample characterization algorithm of a diagnostic laboratory system, the method comprising:
providing an imaging device in the diagnostic laboratory system, wherein the imaging device is controllably movable within the diagnostic laboratory system;
capturing a first image within the diagnostic laboratory system using the imaging device, the first image captured with an imaging condition;
performing an annotation of the first image using an annotation generator of the diagnostic laboratory system to generate a first annotated image; and
updating training of the annotation generator using the first annotated image.

2. The method of claim 1, further comprising:
altering the imaging condition to an altered imaging condition;
capturing a second image within the diagnostic laboratory system using the imaging device with the altered imaging condition;
performing an annotation of the second image using the annotation generator to generate a second annotated image; and
updating the training of the annotation generator using the second annotated image.

3. The method of claim 2, wherein the first image and the second image include a holding location for a sample container.

4. The method of claim 2, wherein the first image and the second image include a sample container.

5. The method of claim 4, further comprising providing a robot comprising a gripper, wherein providing the imaging device comprises affixing the imaging device to the robot, and further comprising gripping the sample container during the capturing of the first image.

6. The method of claim 4, wherein the imaging condition is velocity of the imaging device relative to the sample container during imaging.

7. The method of claim 4, wherein the imaging condition is pose of the imaging device relative to the sample container.

8. The method of claim 4, wherein the imaging condition is a position of the imaging device relative to the sample container.

9. The method of claim 1, wherein the imaging condition is intensity of illumination within the diagnostic laboratory system.

10. The method of claim 1, wherein the annotation is a bounding box or a pixelwise mask of an object in the first image.

11. The method of claim 1, wherein the annotation is one or more properties of a sample container in an image.

12. The method of claim 11, wherein the one or more properties includes sample container orientation with respect to a holding location for a sample handler.

13. The method of claim 11, wherein the one or more properties include at least one of geometry of at least one portion of the sample container, sample container height, sample container diameter, characteristics of a liquid in the sample container, and sample container identification indicia.

14. A method of training a sample characterization algorithm of a diagnostic laboratory system, the method comprising:
providing an imaging device in the diagnostic laboratory system, wherein the imaging device is controllably movable within the diagnostic laboratory system;
capturing a first image of a sample container using the imaging device, the first image captured with a first imaging condition;
performing an annotation of the first image to generate a first annotated image;
altering the first imaging condition to a second imaging condition;
capturing a second image of the sample container using the imaging device with the second imaging condition;
performing the annotation of the second image to generate a second annotated image;
training an annotation generator of the diagnostic laboratory system using at least the first annotated image and the second annotated image;
altering the second imaging condition to a third imaging condition;
capturing a third image of the sample container using the imaging device with the third imaging condition;
performing the annotation of the third image using the annotation generator to generate a third annotated image; and
further training the annotation generator using at least the third annotated image.

15. The method of claim 14, further comprising providing a robot comprising a gripper, wherein providing the imaging device comprises providing the imaging device affixed to the robot, and further comprising gripping the sample container during the capturing of the first image, the second image, or the third image.

16. The method of claim 14, wherein the first imaging condition is a first intensity of illumination illuminating the sample container during capturing the first image, the second imaging condition is a second intensity of illumination illuminating the sample container during capturing the second image, and the third imaging condition is a third intensity of illumination illuminating the sample container during capturing the third image.

17. The method of claim 14, wherein the first imaging condition is a first velocity of the imaging device relative to the sample container during capturing the first image, the second imaging condition is a second velocity of the imaging device relative to the sample container during capturing the second image, and the third imaging condition is a third velocity of the imaging device relative to the sample container during capturing the third image.

18. The method of claim 14, wherein the first imaging condition is a first pose of the imaging device relative to the sample container during capturing the first image, the second imaging condition is a second pose of the imaging device relative to the sample container during capturing the second image, and the third imaging condition is a third pose of the imaging device relative to the sample container during capturing the third image.

19. The method of claim 14, wherein the annotation is a bounding box or a pixelwise mask of the sample container.

20. A diagnostic laboratory system comprising:
an imaging device controllably movable within the diagnostic laboratory system, wherein the imaging device is configured to capture images within the diagnostic laboratory system under different imaging conditions;
a processor coupled to the imaging device;
a memory coupled to the processor, wherein the memory includes an annotation generator trained to annotate images captured by the imaging device, the processor further including computer program code that, when executed by the processor, causes the processor to:
receive first image data of a first image captured by the imaging device using at least one imaging condition;
cause the annotation generator to perform an annotation of the first image to generate a first annotated image; and
update training of the annotation generator using the first annotated image.
